(19) United States
(12) Patent Application Publication
(10) Pub. No.: US 2022/0008521 A1
(43) Pub. Date: Jan. 13, 2022

(54) PHARMACEUTICAL COMPOSITION COMPRISING ANCROD FOR THE TREATMENT OR PROPHYLAXIS OF ENDOCARDITIS

(71) Applicant: Nordmark IP GmbH, Uetersen (DE)

(72) Inventors: Peter VERHAMME, Leuven (BE); Laurens LIESENBORGHS, Leuven (BE)

(73) Assignee: Nordmark IP GmbH, Uetersen (DE)

(21) Appl. No.: 17/294,563

(22) PCT Filed: Nov. 18, 2019
(86) PCT No.: PCT/EP2019/081651

§ 371 Date: May 17, 2021

(30) Foreign Application Priority Data
Nov. 19, 2018 (DE) 10 2018 129 005.5
Mar. 13, 2019 (DE) 10 2019 106 408.2

(51) Int. Cl. A61K 38/48; A61P 31/04; A61P 7/02

(52) U.S. Cl. CPC A61K 38/4806; A61P 7/02; A61P 31/04

(57) ABSTRACT

The present invention concerns ancrod for endocarditis prophylaxis or the treatment of endocarditis, especially for the treatment of damage-induced endocarditis. According to the present invention ancrod is preferably administered by intravenous injection, intravenous infusion, intramuscular injection, subcutaneous injection, intraperitoneal injection or combinations thereof. The present invention also concerns pharmaceutical compositions, especially aqueous solutions or dispersions of ancrod for endocarditis prophylaxis or the treatment of endocarditis.

### INTRODUCTION

The present invention concerns ancrod for endocarditis prophylaxis or the treatment of endocarditis, especially for the treatment of damage-induced endocarditis. According to the present invention ancrod is preferably administered by intravenous injection, intravenous infusion, intramuscular injection, subcutaneous injection, intraperitoneal injection or combinations thereof. The present invention also concerns pharmaceutical compositions, especially aqueous solutions or dispersions of ancrod for endocarditis prophylaxis or the treatment of endocarditis.

### BACKGROUND

Bacterial infection of the heart valves (endocarditis) remains a major clinical problem affecting between 2 and 6 per 100,000 individuals each year and Staphylococcus aureus (S. aureus) is currently the most frequent and most feared causative pathogen (see Hill E E, Herijgers P, Claus P, Vanderschueren S, Herregods M C, Peetermans W E. Infective endocarditis: changing epidemiology and predictors of 6-month mortality: a prospective cohort study. European heart journal; 2007; 28(2); 196-203). Compared to other bacteria S. aureus yields a more destructive and often fatal form of endocarditis. Around 30% to 40% of patients do not survive an endocarditis caused by Staphylococcus aureus (in the following also “S. aureus endocarditis” or “S. aureus infective endocarditis”) despite optimal antibiotic and surgical treatment. With this mortality rate S. aureus endocarditis remains one of the most deadly heart diseases and attempts to prevent the disease have all failed. That is, despite recent progresses in medical technology, the outcome of endocarditis has not improved over the last decades. In addition, attempts to prevent S. aureus endocarditis such as antibiotic and anticoagulation prophylaxis and vaccination have all failed, leaving an unmet need for new therapeutic strategies for this devastating disease (see Federspiel J J, Stearns S C, Peppercorn A F, Chu V H, Fowler V G, Jr. Increasing US rates of endocarditis with Staphylococcus aureus: 1999-2008. Arch Intern Med, 2012; 172(4); 363-5). One of the reasons for the stagnation in the treatment of endocarditis is a lack of understanding of the pathogenesis of endocarditis. Indeed, the real reason why S. aureus is so proficient in causing endocarditis remains elusive.

Ancrod is a peptide derived from the venom of the Malayan pit viper. It acts as defibrinogenating agent. The defribrinogenation of blood results in an anticoagulant effect. Ancrod's anticoagulant effects are thought to derive from the reduction of the fibrinogen concentration in the blood within hours following ancrod administration. Ancrod specifically cleaves only the alpha chain of fibrinogen, resulting in fibrinopeptides A, AP and AY, but not the B-fibrinopeptide. The resulting fibrin polymers are formed imperfectly and much smaller in size (1 to 2 μm long) than the fibrin polymers produced by the action of thrombin. These ancrod-induced microthrombi are markedly susceptible to digestion by plasmin and are rapidly removed from circulation. The blood viscosity in patients receiving ancrod is typically reduced by 30 to 40%. Unlike fibrinolytic agents, ancrod can be administered preoperatively, during an operation or postoperatively.

Ancrod has been indicated or considered to be indicated among others for the treatment of various types of thrombosis and re-thrombosis, priapism, pulmonary hypertension of embolic origin, embolism after insertion of prosthetic cardiac valves and ischemic stroke. It is commercially available from Nordmark Arzneimittel GmbH & Co. KG, Uetersen, Germany.

### Objects of the Present Invention

It is an object of the present invention to provide active pharmaceutical agents for endocarditis prophylaxis. It is further an object of the present invention to provide active pharmaceutical agents for the treatment of endocarditis. It is further an object of the present invention to provide pharmaceutical compositions for endocarditis prophylaxis. And it is an object of the present invention to provide pharmaceutical compositions for the treatment of endocarditis. It is especially an object of the present invention to provide active pharmaceutical agents and pharmaceutical compositions for the prophylaxis and/or the treatment of damage-induced endocarditis.

### DETAILED DESCRIPTION OF THE INVENTION

The present invention concerns ancrod for endocarditis prophylaxis or the treatment of endocarditis. One embodiment of the present invention is ancrod for endocarditis prophylaxis. This embodiment concerns the use of ancrod for the prophylaxis of endocarditis in patients. A further embodiment of the present invention is Ancrod for the treatment of endocarditis. This embodiment concerns the use of ancrod for the treatment of patients with endocarditis. The inventors of the present invention have been able to show that in an animal model (mice, see examples and especially Example 4) ancrod is able to reduce bacterial vegetation (bacterial plaques) on cardiac valves with S. aureus endocarditis. Ancrod is therefore suitable for the prophylaxis and the treatment of endocarditis. In addition, ancrod is a well-known pharmaceutically active substance and its application to endocarditis will pose no problem to the expert in the art.

Without wishing to be bound by this theory, the inventors believe that the effect of ancrod may be based on its action on fibrinogen and/or fibrin. Bacterial vegetation (in the following for short “vegetation”) on cardiac valves with S. aureus endocarditis forms a plaque that comprises S. aureus, platelets and fibrin as components. In this vegetation fibrin may interact with S. aureus, platelets and with the wall surface of the cardiac valves, thereby acting as one component that provides a principal structure for the vegetation or plaque. Ancrod seems to have two possible routes to act on the vegetation or plaque. Firstly, ancrod may be able to act on fibrinogen that is dissolved in blood and reduce the concentration of fibrinogen in the blood. Fibrinogen is the precursor for the fibrin in the vegetation and depletion of fibrinogen in the blood, i.e. decrease of the concentration of fibrinogen in the blood, may prevent the formation of vegetation or plaque on the wall surface of cardiac valves or it may prevent the formation of further volumes of the plaque on plaque already formed on the wall surface of cardiac valves. Secondly ancrod may act directly on the fibrin in the vegetation and may reduce the amount of vegetation on the wall surface of cardiac valves.

A preferred embodiment of the present invention is ancrod for endocarditis prophylaxis or the treatment of endocarditis that is characterized in that the endocarditis is a damage-induced endocarditis. This embodiment concerns the use of ancrod for the prophylaxis of damage-induced endocarditis in patients. This embodiment further concerns the use of ancrod for the treatment of patients with damage-induced endocarditis.

In the classical view, endocarditis is caused when valvular endothelium is disrupted on damaged cardiac valves. This exposes the subendothelial matrix of the valves and causes local fibrin deposition to which bacteria can adhere (Dayer M J, Jones S, Prendergast B, Baddour L M, Lockhart P B, Thornhill M H. Incidence of infective endocarditis in England, 2000-13: a secular trend, interrupted time-series analysis. The Lancet; 2015; 385(9974); 1219-28). Nevertheless, 40% of patients with native valve endocarditis have intact heart valves before infection, so cardiac valve damage is absent in these patients. The infective lesions in these patients are thought to occur on inflamed rather than on damaged heart valves. During inflammation, activated endothelial cells express various molecules to which bacteria can adhere and change from an anticoagulant to a prothrombotic state, allowing coagulation on their surface, creating early vegetation. The inventors therefore hypothesize that there are two independent risk states that can lead to endocarditis: cardiac valve damage and cardiac valve inflammation. In damage-induced endocarditis the valvular endothelium of the cardiac valves is disrupted or damaged and part of the subendothelial matrix of the cardiac valves is exposed. In inflammation-induced endocarditis the valvular endothelium of the cardiac valves is typically intact but the endothelial cells are activated and therefore altered by inflammation. This activation promotes formation and/or adhesion of bacterial vegetation. Therefore the endothelial cells form the basis for the bacterial vegetation in inflammation-induced endocarditis. However, these two mechanisms may not be mutually exclusive and may overlap to some degree. Therefore treatment for damage-induced endocarditis may also be effective against inflammation-induced endocarditis to some extent and vice versa.

Obviously cardiac valve damage may dominate when the valve is damaged directly by surgical procedure for example by use of a catheter. Clinical scenarios in which cardiac valve damage dominates are those involving patients with rheumatic and congenital valve disease or aortic stenosis. In these cases turbulent blood flow damages the endothelium and predisposes to endocarditis.

On the other hand examples of patients at risk for inflammation-induced endocarditis are for example intravenous drug users, which are mostly young people without valve abnormalities, but are in a constant inflammatory state owing to the injections of contaminated materials, intensive care patients who develop an S. aureus-associated catheter infection or intensive care patients with nosocomial bacteremia. In addition, S. aureus itself can trigger endothelial cell activation, thereby facilitating its adhesion and inflammation-induced endocarditis.

A further preferred embodiment of the present invention is ancrod for endocarditis prophylaxis or the treatment of endocarditis that is characterized in that the endocarditis is a damage-induced endocarditis in patients with a disease selected from the group consisting of valve damage by surgical procedure for example by use of a catheter in the aortic valve, congenital heart disease and aortic stenosis. This embodiment concerns the use of ancrod for the prophylaxis or the treatment of damage-induced endocarditis in patients with a disease selected from the group consisting of valve damage by surgical procedure for example by use of a catheter, congenital heart disease and aortic stenosis where turbulent flow creates valve damage. Especially preferred is ancrod for endocarditis prophylaxis or the treatment of endocarditis, wherein the endocarditis is a damage-induced endocarditis and wherein the damaged is induced by surgery. Most preferred is ancrod for endocarditis prophylaxis or the treatment of endocarditis, wherein the endocarditis is a damage-induced endocarditis and wherein the damage is induced by surgery by use of a catheter in the aortic valve. Preferred is further ancrod according to the present invention, characterized in that the endocarditis is a damage-induced endocarditis, wherein the damage is induced by cardiac catheter examination.

A further embodiment of the present invention is ancrod for endocarditis prophylaxis or the treatment of endocarditis that is characterized in that the endocarditis is an inflammation-induced endocarditis. This embodiment concerns the use of ancrod for the prophylaxis of inflammation-induced endocarditis in patients. This embodiment further concerns the use of ancrod for the treatment of inflammation-induced endocarditis in patients. Preferred in this embodiment is ancrod for endocarditis prophylaxis or the treatment of endocarditis that is characterized in that the endocarditis is an inflammation-induced endocarditis in patients which are intravenous drug users, in intensive care patients who develop S. aureus-associated catheter infections or in intensive care patients with nosocomial bacteremia. This embodiment concerns the use of ancrod for the prophylaxis or the treatment of inflammation-induced endocarditis in patients which are intravenous drug users, intensive care patients who develop S. aureus-associated catheter infections or intensive care patients with nosocomial bacteremia or in bacteremic patients.

The patients treated with the inventive ancrod are mammals and preferably humans.

Ancrod of the present invention is preferably administered by a method selected from the group consisting of injection directly into the vascular system, infusion directly into the vascular system, injection into tissue, injection into the peritoneal cavity of the abdominal cavity and combinations thereof. Ancrod is preferably administered by a method selected from the group consisting of intravenous injection, intravenous infusion, intramuscular injection, subcutaneous injection, intraperitoneal injection and combinations thereof. Among them intravenous injection, intravenous infusion and combinations thereof are even more preferred. Most preferred is intravenous injection. Here administration routes that instantly provide an effective blood concentration of ancrod can be used advantageously in combination with those administration routes that show a slower increase in blood level. Hence, administration via injection directly into the vascular system—especially by intravenous injection—can be used to provide an effective level of ancrod in the blood stream instantly, whereas application by intravenous infusion, intramuscular injection, subcutaneous injection, intraperitoneal injection and combinations thereof may serve to slowly gain and then maintain a rather constant and effective concentration of ancrod in the blood stream for a longer period of time.

A preferred embodiment of the present invention is ancrod for endocarditis prophylaxis or the treatment of endocarditis, characterized in that ancrod is administered in an amount of from 0.1 to 10 international units (iU) per kg of body weight, especially preferred in an amount of from 0.5 to 5 iU per kg and even more preferred in an amount of from 1 to 2 iU per kg. The standard for the quantitative measurement of the enzymatic activity of ancrod in international units as used herein is disclosed in document WHO/BS/2016.2282 of the World Health Organization.

A further embodiment of the present invention is a pharmaceutical composition comprising ancrod for endocarditis prophylaxis or the treatment of endocarditis. The pharmaceutical composition may further comprise further active agents and excipients. The pharmaceutical composition is preferably an aqueous solution or dispersion. The pharmaceutical composition may also be a gel or solid that may be dissolved or dispersed in water or other solvents to form a solution or a dispersion.

### BRIEF DESCRIPTION OF THE FIGURES

The figures show the following:

FIG. 1: Adhesion of wild type S. aureus on damaged and undamaged cardiac valves

FIG. 2: Adhesion of a clinical strain of S. aureus on damaged cardiac valves dependent on the amount of damage.

FIG. 3: Scanning electron microscopy image of an intact aortic valve (162×)

FIG. 4: Scanning electron microscopy image of a valve with damage-induced endocarditis (162×)

FIG. 5: Scanning electron microscopy image of a valve with damage-induced endocarditis (9140×)

FIG. 6: Effect of ancrod on the amount of vegetation produced by adhesion of S. aureus to the inner walls of cardiac valves at a dosage of 6 iU

### EXPERIMENTAL PART

### Methods

### Bacterial Strains

The reference strains used in this study were S. aureus Newman, originally isolated from a case of osteomyelitis, and S. aureus USA300. Clinical strains of S. aureus were isolated from blood cultures of patients from the University Hospitals of Leuven. Before usage the bacteria were grown overnight in Tryptic Soy Broth (Sigma-Aldrich, Darmstadt, Germany) at 37° C., washed twice in phosphate-buffered saline (PBS) and quantified by recording optical densitometry at a wavelength of 600 nm (OD600). The inoculum size was verified by plating on blood agar.

### Animals

Mice of the wild type (WT) C57BL/6 were used. Before surgery mice were anesthetized with ketamine (125 mg/kg body weight) and xylazine (12.5 mg/kg body weight) and anesthesia was checked by pedal reflex. Mice that were not immediately sacrificed after the procedure also received buprenorphine (0.1 mg/kg body weight) subcutaneously 20 to 30 min prior to the surgery and twice daily thereafter.

### Spontaneous Endocarditis

To see whether mice develop endocarditis spontaneously, old mice (>18 months of age and 27 months of age) and young mice (10 to 15 weeks of age) were injected via tail vein with 2×10⁶ to 3×10⁷ CFU S. aureus Newman. Mice were sacrificed three days after injection and the hearts were excised, fixed overnight in paraformaldehyde 4% and embedded in paraffin. Sections of the aortic valve were stained with Brown-Hopps tissue Gram staining and imaged with light microscopy (Axiovert 200 m, Carl-Zeiss, Oberkochen, Germany). A researcher analyzed the sections for presence of endocarditis without knowledge of the treatment conditions.

### Endocarditis Mouse Model to Study Early Bacterial Adhesion

Bacteria were fluorescence-labeled with 5(6)-carboxyfluorescein-N-hydroxysuccinimidylester (Sigma-Aldrich, Darmstadt, Germany; 30 μg/mL) or Texas Red®-X, succinimidyl ester (Thermo Fisher, Waltham, Mass., USA; 10 μM) for 45 minutes before usage. The mice were anesthetized as described above and injected with 3×10⁷ CFU fluorescent S. aureus bacteria via tail vein. Subsequently, the carotid artery was dissected and a 32-gauge polyurethane catheter was inserted (RecathCo, Allison Park, Penn., USA). The catheter was moved upstream beyond the aortic valve until pulsation of the catheter was detected, assuring its position in the left ventricle, beyond the aortic valve. The time between injection of the bacteria and placement of the catheter was approximately 15 minutes.

To generate endothelial damage for the examination of the effect of ancrod, the catheter was left in place during 15 or 30 minutes, damaging the valves with every heartbeat. Afterwards, the catheter was removed and the carotid artery ligated to prevent blood loss. The control group consisted of mice that underwent sham operation: the catheter was placed as described above, but immediately removed and the carotid artery was ligated.

After removal of the catheter, the mice were immediately sacrificed and transcardially perfused with saline and paraformaldehyde 4% both for two minutes. The hearts were then fixed in paraformaldehyde 4% overnight, transferred to sucrose 25%, embedded and frozen in Tissue-Tek o.c.t. compound (commercially available from Sakura Finetek Europe B.V., in Alphen aan den Rijn, The Netherlands). Afterwards, 4 to 7 cryosections, 200 μm in thick, covering the entire aortic valve were made. Of each 200 μm in section a Z-stack was made with confocal microscopy (LSM 700 or LSM880 Carl-Zeiss, Oberkochen, Germany) as to image the entire aortic valve. These images were then analyzed in 3D with Imaris (Bitplane, Zurich, Switzerland) or Image J (Image J, NIH, Bethesda, USA).

### Studying Long-Term Endocarditis Development

In these experiments non-fluorescent bacteria (2×10⁶ to 2×10⁷ CFU/mouse) were injected and a catheter cardiac valve damage was induced as described above. Afterwards, the catheter was removed, the carotid artery was ligated and the skin closed. The mice recovered from surgery and were monitored for wellbeing four times per day, up to day three, after which they were sacrificed. Hearts were excised, fixed overnight in paraformaldehyde 4% and embedded in paraffin. Sections of the aortic valve were stained with Brown-Hopps tissue Gram staining and imaged with light microscopy (Axiovert 200 m, Carl-Zeiss, Oberkochen, Germany). A researcher analyzed the sections for presence of endocarditis without knowledge of the experimental conditions.

### Electron Microscopy

Aortic valves were dissected and fixed with 2% paraformaldehyde, 2.5% glutaraldehyde and 0.02% sodium azide mixture in 0.05 M sodium cacodylate buffer. After double treatment with osmium tetroxide and thiocarbohydrazide with OTO protocol (Friedman P L, Ellisman M H. Enhanced visualization of peripheral nerve and sensory receptors in the scanning electron microscope using cryofracture and osmium-thiocarbohydrazide-osmium impregnation. J Neurocytol; 1981; 10(1); 111-31) samples were dehydrated in ethanol followed by hexamethyldisilazane, dried, mounted on aluminium stubs, sputter coated with chromium and examined in a digital field emission scanning electron microscope (Carl Zeiss) at 5 kV accelerating voltage.

### Statistical Analysis

All calculations were done with GraphPad Prism 5.0 d (GraphPad Software, La Jolla, Calif., USA). If normally distributed the two-tailed Student's t-test was used. The early adhesion measurements were found to be skewed and were log transformed, after which t-testing could be applied. All values are reported as mean±standard deviation (SD). To test proportion (endocarditis or not), the Fisher's exact test was used. A P-value of <0.05 was considered statistically significant.

### EXAMPLES

### Example 1: Spontaneous Occurrence of Infective Endocarditis in Mice

The validity of mice to study infective endocarditis and whether endocarditis spontaneously occurs in bacteremic mice was studied in a first experiment. 10 young mice (10 to 15 weeks old) were injected intravenously with 2×10⁶ CFU of S. aureus Newman. None of them developed endocarditis. The experiment was repeated with 3×10⁷ CFU with the same result. However, when 10 old mice (>1.5 years of age and 27 months of age) were injected with 2×10⁶ CFU S. aureus Newman, two out of ten developed endocarditis, without any preceding surgical procedure. These lesions shared remarkable similarity with human endocarditis lesions, consisting of large bacterial colonies growing unimpeded in a meshwork of platelets and fibrin, mostly devoid of leukocytes.

Mice are therefore in principle suitable as a model for infective endocarditis.

### Example 2: Early Bacterial Adhesion to the Heart Valves

Mice were injected intravenously with fluorescence-labeled S. aureus and bacterial adhesion to the valve leaflets was quantified using 3D confocal microscopy on 200 μm thick cryosections of the aortic valves. A first group of mice only received an injection of S. aureus and underwent no manipulation of the valves, representing the bacteremic patient without risk factors. In a second group, cardiac valve damage was simulated by insertion of a 32-gauge polyurethane catheter in the carotid artery, which was advanced beyond the aortic valve. The catheter was left in place damaging the valve during 15 minutes, after which it was removed.

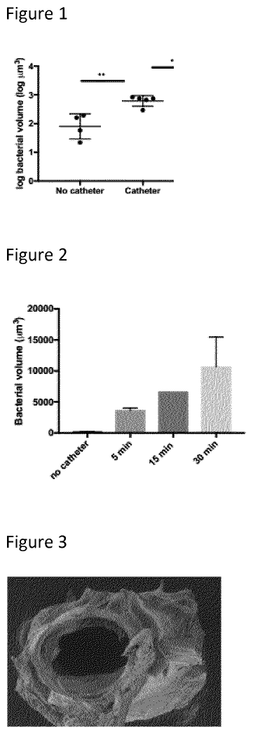

FIG. 1 shows the result. As can be seen by the common logarithm of the volume of the bacterial vegetation, in the bacteremia group without manipulation of the aortic valve, hardly any bacteria adhered to the valves (see entries for “No catheter” in FIG. 1). However, in mice where the cardiac valves were damaged, a significant increase in bacterial adhesion was seen (P<0.01; see entries for “Catheter” in FIG. 1). These results were confirmed with a clinical strain, which was a CC5-positive S. aureus from an endocarditis patient; the longer the cardiac valves were damaged, the stronger the clinical strain adhered (see FIG. 2, here the volume of the bacterial vegetation is shown, not its common logarithm). Similar results were obtained with the S. aureus USA300-strain (data not shown).

### Example 3: From Early Bacterial Adhesion to a Mature Endocarditis

In a next step it was tested, whether this observed early bacterial adhesion was indeed the first step in the development of a mature endocarditis. To this end, again an intravenous injection with S. aureus was conducted and a 32-gauge polyurethane catheter was used to induce cardiac valve damage (for 30 minutes this time). Afterwards, the catheter was immediately removed and the mice were followed over a three-day period. Sections of the aortic valve were analyzed at various time points. Ten mice for each experiment were used.

None of the control mice (sham operation) developed endocarditis after injection of 2×10⁶ CFU S. aureus. In contrast, in some mice where the aortic valves were damaged during 30 minutes, endocarditis did occur: 0/5 (0%) for S. aureus Newman, 1/12 (8%) for S. aureus USA300 and 1/11 (9%) for the clinical strain (P>0.05 for all experiments vs. sham operation). However, when a higher bacterial load was used (2×10⁷ CFU) a larger proportion of mice developed endocarditis: 2/6 (33%) for S. aureus Newman, 1/12 (8%) for S. aureus USA300 and 8/10 (80%) for the clinical strain, which was a CC5-positive S. aureus from an endocarditis patient (P=0.45, P=0.9, P<0.01 vs. sham operation for Newman, USA300 and the clinical strain respectively).

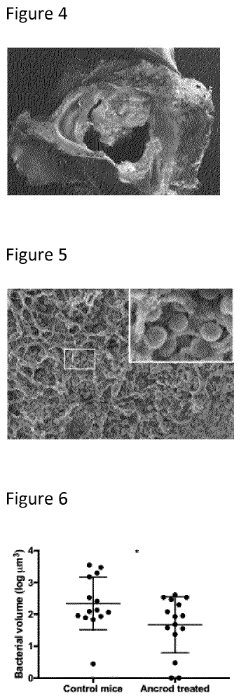

Using light microscopy the different stages in the development of these endocarditis lesions could be captured, observing how small, early lesions would grow to large, destructive vegetation that ultimately destroyed the whole aortic valve. Importantly, these experimental vegetation looked very similar to those that spontaneously occurred in older mice as described in Example 1. Echocardiography confirmed that these vegetation originated from the aortic valve and could in some cases cause serious aortic regurgitation. In addition, electron microscopy was used to study these lesions in more detail (FIGS. 3 to 5), revealing individual Staphylococci in large destructive vegetation consisting of platelets and fibrin. FIG. 3 shows a scanning electron microscopy image of an intact aortic valve. However, FIGS. 4 and 5 show a valve damaged by endocarditis. FIG. 4 is the picture of a valve in the same size as that of FIG. 3 and shows in comparison to FIG. 3 that a large amount of vegetation is produced. In a larger magnification FIG. 5 shows individual Staphylococci in the vegetation of the cardiac valve shown in FIG. 4. It is therefore apparent, that the induced endocarditis develops upon time into a full-blown endocarditis.

### Example 4: Effect of Ancrod on Bacterial Adhesion

Experiments as described in Example 2 were conducted. However, as a first step C57BL/6-mice were injected intravenously with 6 international units (iU) per mouse of ancrod (Nordmark Arzneimittel GmbH & Co. KG, Uetersen, Germany) before the experiments. The concentrations used in this example are much higher than the preferred dose range for a therapeutic use in humans. The reason is that the present examples serve as a proof of concept. In a control group ancrod was not used. Instead an equal volume of saline was administered as placebo. 5 to 7 hours after the administration of ancrod or placebo, respectively, 3×10⁷ CFU of fluorescence-labeled Staphylococcus aureus Newman were injected intravenously into the mice and a 32-gauge polyurethane catheter was inserted in the carotid artery and advanced beyond the aortic valve. To create cardiac valve damage, the catheter was left in place for 15 minutes. Thereafter the mice were immediately sacrificed and bacterial adhesion was measured with confocal microscopy.

FIG. 6 shows a comparison of the results for ancrod-treated mice vs. control mice, which were not treated with ancrod. The results in FIG. 6 represent vegetation volumes transformed by the common logarithm, every dot corresponds to a single mouse. Mean±standard deviation range is given, *p<0.05, two-tailed unpaired Student's t-test. As can clearly be seen, mice treated with ancrod showed significantly reduced bacterial adhesion (P<0.05).

1. Ancrod for endocarditis prophylaxis or the treatment of endocarditis.

2. Ancrod according to claim 1 for endocarditis prophylaxis.

3. Ancrod according to claim 1 for the treatment of endocarditis.

4. Ancrod according to claim 1, wherein the endocarditis is a damage-induced endocarditis.

5. Ancrod according to claim 1, wherein ancrod is administered by a method selected from the group consisting of injection directly into the vascular system, infusion directly into the vascular system, injection into tissue, injection into the peritoneal cavity of the abdominal cavity and combinations thereof.

6. Ancrod according to claim 1, wherein ancrod is administered by a method selected from the group consisting of intravenous injection, intravenous infusion, intramuscular injection, subcutaneous injection, intraperitoneal injection and combinations thereof.

7. Ancrod according to claim 1, characterized in that ancrod is administered by a method selected from the group consisting of intravenous injection, intravenous infusion and combinations thereof.

8. Ancrod according to claim 1, characterized in that ancrod is administered by intravenous injection.

9. Ancrod according to claim 1, characterized in that ancrod is administered in an amount of from 0.1 to 10 iU per kg of body weight.

10. Ancrod according to claim 1, characterized in that ancrod is administered in an amount of from 0.5 to 5 iU per kg of body weight.

11. Ancrod according to claim 1, characterized in that ancrod is administered in an amount of from 1 to 2 iU per kg of body weight.

12. Ancrod according to claim 1, characterized in that the endocarditis is a damage-induced endocarditis, wherein the damage is induced by surgery.

13. Ancrod according to claim 1, characterized in that the endocarditis is a damage-induced endocarditis, wherein the damage is induced by cardiac catheter examination.

14. A pharmaceutical composition comprising ancrod according to claim 1.

15. A method for endocarditis prophylaxis or the treatment of endocarditis, comprising:
administering to a patient in need thereof an effective amount of ancrod.

16. The method according to claim 15, wherein the method is for endocarditis prophylaxis.

17. The method according to claim 15, wherein the method is for the treatment of endocarditis.

18. The method according to claim 15, wherein the endocarditis is a damage-induced endocarditis.

19. The method according to claim 15, wherein the administering is performed by a method selected from the group consisting of injection directly into the vascular system, infusion directly into the vascular system, injection into tissue, injection into the peritoneal cavity of the abdominal cavity and combinations thereof.

20. The method according to claim 15, wherein the administering is performed by a method selected from the group consisting of intravenous injection, intravenous infusion, intramuscular injection, subcutaneous injection, intraperitoneal injection and combinations thereof.

21. The method according to claim 15, wherein the ancrod is administered in an amount of from 0.1 to 10 iU per kg of body weight.
